(19) United States
(12) Patent Application Publication
(10) Pub. No.: US 2024/0277333 A1
(43) Pub. Date: Aug. 22, 2024

(54) APPARATUSES, SYSTEMS, AND METHODS FOR FASCIAL CLOSURE

(71) Applicant: Regents of the University of Minnesota, Minneapolis, MN (US)

(72) Inventors: Bryan Ladd, Minneapolis, MN (US); Hannah Sheridan, Minneapolis, MN (US); Vibha Mavanji, Minneapolis, MN (US); Grant Ryerse, Minneapolis, MN (US); Rick Marcusen, Minneapolis, MN (US); Rohan Thakur, Minneapolis, MN (US)

(21) Appl. No.: 18/571,000

(22) PCT Filed: Jun. 14, 2022
(86) PCT No.: PCT/US2022/033412

§ 371 Date: Dec. 15, 2023

Related U.S. Application Data
(60) Provisional application No. 63/202,540, filed on Jun. 15, 2021.

(51) Int. Cl. A61B 17/04; A61B 17/00; A61B 17/06

(52) U.S. Cl. CPC A61B 17/0487; A61B 17/06066; A61B 2017/00004; A61B 2017/06176

(57) ABSTRACT

A biocompatible device for fascial closing following surgeries and a corresponding hand-held tensioning mechanism. The device is modeled after a zip tie and consists of a needle, body, and locking mechanism. The needle and body are driven through fascia, then the needle is separated to allow for the feeding of the body through the locking mechanism to provide tension against fascia separation. The body of the tie comprises a first face with a series of recessed teeth and a second face with a substantially smooth surface. When the tie forms a loop in a locked configuration, the first face of the body is oriented toward an internal volume of the loop and in direct contact with a target, whereas the second face of the body is oriented towards surrounding tissues that minimizes trauma to surrounding tissues.

### PRIORITY CLAIM

The present application claims priority to United States Provisional Application Serial No. 63/202,540, filed June 15, 2021 and entitled “APPARATUSES, SYSTEMS, AND METHODS FOR FASCIAL CLOSURE”, which application is hereby incorporated by reference in its entirety.

### TECHNICAL FIELD

Embodiments described herein relate to internal fixation devices, including fasteners and fixators. Such fixation devices and systems can include flexible bands or straps.

### BACKGROUND

The foundation of good wound closure starts with a good fascial closure. Fascia is a band of connective tissue beneath the skin that attaches, stabilizes, and separates organs and skin within the body. The most prevalent method used to achieve fascial closure is by hand tying sutures along the length of the wound. Unfortunately, complications occasionally exist associated with poor closure of the galea, even in the most skilled of hands. These complications can include wound dehiscence, muscle retraction, herniation, poor cosmetic wound healing, protruding knots, and a route for deep wound infections. Complications become more likely in patient populations that undergo frequent repeat operations (e.g., trauma, oncology, shunted hydrocephalus). For these patients, the fascia typically is lost to scar tissue formation and the overlying skin thins, increasing the difficulty to achieve a robust closure. Sutures placed in the location of the fascia are prone to “pulling through” when tension is applied. This term refers to what occurs when the sutures break through the hole punctured in the fascia before bringing each side together. Alternative suturing techniques, such as full thickness vertical mattress sutures, can be used in some circumstances to close these friable tissues, but these alternatives increase operative time and are of questionable benefit. Regardless of the complexity of a wound, all sutures must be tied to a similar tension. If there is too much variance in tension between sutures, sutures under high tension are prone to breakage or early loss of tensile strength. This leaves a segment of the wound susceptible to wound dehiscence due to the loss of the integrity provided by the underlying fascial closure.

It is often the case that a fascial suture is tightened as much as possible by a medical practitioner, to the point that the suture is on the verge of breaking. Different types of suture material are used for different purposes, such as braided or monofilament fibers having different cross-sectional areas for varied surface characteristics and tensile strength. Additionally, it may be necessary to apply sutures to pull together the edges of an incision by an initial amount, and then later in the same procedure re-tie the sutures or add new sutures to pull the edges closer together. This reduces the force that is needed on the suture in the first pass, and therefore reduces the hand strength needed by the medical professional and reduces the possibility for injury caused by the suture material itself pulling on the patient. In general, the manual nature of the suture tying leaves patients vulnerable to human variation.

Fascial sutures are placed via a repeatable process one at a time. First, the needle is grasped by the needle driver on the needle body. Once the tissue being driven into has been stabilized with forceps, the needle is penetrated through the tissue at an approximately 90° angle typically between 1-3 mm from the fascia edge, depending on the thickness. Next, the needle and suture are pulled through the puncture hole and the needle penetrates the other side of the wound perpendicular to the fascia, on the other side of the wound. The surgeon then brings the tissue together by pulling on either side of the suture, applying the tension by hand. Once the surgeon is satisfied with the degree of closure, they place a knot to secure the suture and lock the tissue in place. Multiple knots are often thrown to ensure reliable locking. The process can take up to 30 or 40 minutes for some procedures.

Cable ties, sometimes referred to as zip ties, are commonly used flexible fasteners that consist of a notched body with a locking mechanism on one end that the other end of the body can be looped through. As the body is pulled through the locking mechanism, small notches/teeth within it match the geometry of the body's notches, and engage to ratchet the body through, preventing reverse motion beyond the most recent tooth to pass through the ratchet. Pulling the body through tightens the zip tie loop and can be done by hand, or with a zip tie tensioning device which can tighten the zip tie to a desired degree of tension and cut the excess zip tie material flush with the locking mechanism.

Cable ties have been disclosed for use with bone or cartilage, such as in U.S. Patent Publication No. 2013/0261625 to Koch. The ties disclosed in Koch provide ratcheting mechanisms and are shaped to correspond to the boney structures that they wrap around. The tensioning device for a cable tie, which is often shaped like a gun and referred to as a “zip tie gun,” requires the user to pull a trigger that ratchets the zip tie tight around an object. This is a knotless securing method with little to no tension variance between ties, ensuring a uniformly tight closure. The notch engagement design, which only allows translational movement forwards through a lock to increase and hold tension has been emulated in a few surgical closures, such as those described in U.S. Pat. No. 7,972,347 B2 to Garvin and U.S. Pat. No. 9,757,131 to Sanders. Garvin describes a single piece surgical wound fastener that clasps with a male connecting strap and female connector to be bidirectionally ratcheted with a clamp to tighten the wound closure. The device is directed to be affixed to the tissue directly on either side of the wound with barbed insertion tongues and intended to provide even tensioning. Sanders describes a strap tie assembly ratcheting via a plurality of engagements in a receiving channel which is affixed from large bases attached directly to the skin with barbs or hooks. Other ratcheting devices for attachments to bone are described in, for example, U.S. Pat. No. 8,845,686 to Bennett and the related commercial products sold by ZipTek LLC.

The cable ties described in Koch, Garvin, Sanders, and Bennett are not suitable as suture replacements for a variety of reasons. First, sutures should not need to be removed in the majority of applications. That is, sutures typically dissolve in the body in about the time it takes for the incision to heal (or longer). Otherwise, it would be necessary to reopen the incision to remove the sutures, which would defeat the purpose of the procedure. For that reason, ties that are used for connecting bone are not usable for tissue repair such as sutures for the fascia. For example, Koch describes use of PEEK or PEKK, neither of which would dissolve, while Bennett uses metallic screws for anchoring. While the structures and materials of these references are therefore suitable for bone and cartilage procedures, they cannot be incorporated into a tissue repair application.

Patients who have wounds closed poorly or have pieces of the closure device remaining may experience poor wound closure, wound dehiscence, muscle retraction or spasms, herniation, and cosmetic problems. Additionally, remaining material can provide a route for deep infections. Therefore it is important to properly close a wound, preferably using the least amount of time and leaving little to no material behind (such as by using dissolvable materials).

### SUMMARY

The systems, devices, and methods described herein add ratcheting functionality for tissue repair. This reduces the time needed to carry out a procedure, is safer and more comfortable for physicians, reduces or eliminates the chance for breakage of the suture, and permits tightening and re-tightening of the same ties. The ties disclosed herein have a variety of shapes in different embodiments that are relatively low-profile and that will provide adequate holding strength for an incision to repair itself, while also being dissolvable for long-term patient safety and comfort.

According to an embodiment, a tie is made of a biodegradable material. The tie includes a body extending a long a length from a proximal end to a distal end, a needle coupled to the body at the distal end, and a locking mechanism. The locking mechanism is coupled to the body at the proximal end and oriented to receive the distal end of the body, such that the body can be pulled through the locking mechanism to create a loop. The body includes a series of recessed teeth defined within an otherwise smooth form factor, wherein each tooth of the series of recessed teeth includes a base and an apex, with the base extending into the body and the apex flush with a top face of the body defined by the form factor, such that each tooth has a cross-section in the shape of a right triangle, and wherein each tooth faces inwards when the body is arranged in the locking mechanism to form the loop.

According to another embodiment, a tie is made of a biodegradable material and includes a body extending a long a length from a proximal end to a distal end, a needle coupled to the body at the distal end, and a locking mechanism coupled to the body at the proximal end and oriented to receive the distal end of the body, such that the body can be pulled through the locking mechanism to create a loop, wherein the body comprises ramp extrusions that make up a series of teeth each having a triangular cross-section, wherein a width of each tooth is equal to a width of the tie, and wherein each tooth faces inwards when the body is arranged in the locking mechanism to form the loop.

In either of the first two embodiments, the locking body can define a receiving channel, and wherein the receiving channel is configured to receive the body. The tie can include a locking engagement arranged in the receiving channel and responsive to an applied force of a tie passing through to bend from a hinge towards the sidewall of the lock, wherein the locking engagement is configured to mate with the teeth with a responsive force, fully grasping a tooth and restricting translational movement backwards through the receiving channel. The extension of the locking device beyond a closure site can be minimized. The body can be passed through the lock is ratcheted to hold a target tension.

The biodegradable material can be biocompatible and bioabsortive in a body over a target time period. The biodegradable material further can be a compositional mixture of PLA and PLGA from 0:200 to 200:0. The material can enable the tie to be closed to form a loop of a target diameter. The needle can further have a diameter, an inner radius, and an outer radius, wherein the diameter is equal to or greater than a width of the body, the inner radius configured to match a bottom face of the body, and the outer radius configured to match with a teeth side of the body.

The material can enable the zip-tie to be cut with standard Operating-Room scissors. The tie can have a surface area configured to be directly applied against the body and better supports surgical closure. The tie can be used for surgical closure in a procedure such as to stitch through fascial layers; to perform spinal closures to support the high tensile strength and force needed to hold closed the wound; cranial closures; laparoscopic surgery; endoscopic surgery; to stitch bones together; sternum closure in open heart and other applicable surgeries; and emergency situations and applications by the military.

According to another embodiment, a method for surgical fascial closure includes securing a tie with surgical drivers, the tie comprising a body, needle coupled to the body at a distal end; the body extending a long a length from the distal end to a proximal end, a locking mechanism coupled to the body at the proximal end and oriented to receive the distal end of the body, such that the body can be pulled through the locking mechanism to create a loop, wherein the body further comprises ramp extrusions that make up a series of teeth each having a triangular cross-section, wherein a width of each tooth is equal to a width of the tie, and wherein each tooth faces inwards when the body is arranged in the locking mechanism to form the loop; driving the tie through fascial layers, with the surgical drivers, at a distance of X to Y from a first side of a wound, and up through the fascial layers at second side of the wound at the same distance X to Y from the wound, where X to Y are defined by dimensions of the tie used; and passing the distal end of the body through the locking mechanism.

The locking body mechanism can include a receiving channel, and the receiving channel can be configured to receive the body and is marginally larger than the body to account for a close fit. The method can include using a device having a locking engagement arranged in the receiving channel and responsive to an applied force of a tie passing through to bend from a hinge towards the sidewall of the lock, wherein the locking engagement is configured to mate with the teeth with a responsive force, fully grasping a tooth and restricting translational movement backwards through the receiving channel. The method can include using the applied force to ratchet the ties placed along the length of the wound towards closure, either one by one, or gradually, moving from one to the next until a target tension is achieved; and cutting the tie body flush with the lock at that tension. The needle can be cut off from the body of the tie before securing the tie. Securing the tie with the surgical drivers can involve securing the needle with the surgical drivers, and driving the tie through the facial layers comprises driving the needle through the facial layers.

According to another embodiment, a tie tensioning gun can be specialized for use with surgical ties. The tie tensioning gun can include an external frame encasing the internal components with an opening in the side to load a tie; a clamping mechanism for gripping the tie in the opening of the device; a handle, formed as a trigger, that is pulled to clamp and tighten the tie; and at least three internal links comprising a mechanism that translates a rotational motion, created on the handle when pulled, to linear motion.

The tie tensioning gun can further include a spring with an adjustable length to allow for cutting the tie at a predetermined tension, and a blade, wherein one of the at least three links has a proximal end and the blade is couple to the proximal end of the one of the at least three links. The blade can cut an excess of the tie when a locking mechanism on a proximal end of the tie is flush with the opening in the side of the external plastic frame. The tie tension gun can include a container configured to receive the excess of the tie when cut.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 is a detailed cross-sectional view of a fascia that can be held by devices described herein.

FIGS. 2A and 2B are perspective and top views, respectively, of a fastener according to a recessed-tooth embodiment.

FIG. 3 is a perspective view of the recessed-style fascia closure device of FIGS. 2A and 2B, further depicting the relative heights of each component, where the needle is the largest component.

FIG. 4 is a cross sectional view of the recessed-style zip tie body component of FIGS. 2A and 2B, illustrating the shape and spacing pattern of the teeth notched into the body.

FIG. 5 is a perspective view of the recessed-style locking mechanism component, illustrating the primary four bodies of that component in the recessed-style embodiment of FIGS. 2A and 2B.

FIG. 6 is a cross sectional view of the recessed-style locking mechanism component of FIGS. 2A and 2B, depicting the relative size of the internal body that allows for ratcheting motion of the zip tie through the component.

FIG. 7 is a cross sectional view of the recessed-style device of FIGS. 2A and 2B being fed through the locking mechanism, illustrating how the tongue of the locking mechanism fits in with the device body to prevent backwards motion.

FIG. 8 is a top plan view of the recessed-style locking mechanism component of FIGS. 2A and 2B, illustrating the relative body size of the major components.

FIGS. 9-15 depict a fascia closure device with protruding teeth, according to another embodiment.

FIGS. 16-21 depict a fascia closing device having a circular cross-sectional shape according to another embodiment.

FIGS. 22 and 23 depict a side-loaded embodiment, which can have a reduced profile.

FIG. 24 is a perspective view of a needle that can be used with any of the embodiments described above.

FIGS. 25A and 25B are a perspective and bottom view, respectively, of an alternative recessed style locking mechanism, according to embodiments.

FIGS. 26A-26D are a side, bottom, top, and perspective view of a fascia closure device incorporating the alternative recessed style locking mechanism of FIGS. 25A and 25B.

FIG. 27 is a near view of a recessed portion of the alternative recessed style locking mechanism of FIGS. 25A and 25B.

FIG. 28 is a cutaway view of the recessed portion of FIG. 27.

FIG. 29 depicts the internal conformation of the recessed portion of FIGS. 27 and 28.

FIG. 30 is a fascia closure device incorporating an alternative locking mechanism, according to embodiments.

FIGS. 31A and 31B are side views of the two interlocking ends of the fascia closure device of FIG. 30.

FIGS. 32A and 32B are a bottom perspective and plan view, respectively, of the locking mechanism of the fascia closure device of FIG. 30.

FIG. 33 is a top plan view of the fascia closure device of FIG. 30, focusing on the end with the locking mechanism and showing the teeth distributed along the length of the device.

FIG. 34 depicts the interaction of the interlocking ends of FIGS. 31A and 31B.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

Tissue repair, including fascia repair, relies in large part on the use of sutures that can easily break, cause injury to the medical practitioner's hands during install due to the force required, and that are time-intensive to make. Ratcheting functionality reduces the potential for injury or discomfort for the medical team, and speeds up the process of closing an incision. The total number of ratcheting bands can be reduced as compared to sutures because the bands are larger and sturdier than sutures. The bands can also have significantly higher tensile loads applied due to their increased cross-sectional area.

Unlike existing ratcheting devices such as zip-ties, in suture replacement it is beneficial to use materials that dissolve over time. The dissolving time can be pre-set by using different materials, shapes, and sizes. Additionally, the bands disclosed herein can have different sizes or cross-sectional areas to set the maximum level of tensile force that can be supported. As such, larger or stronger patients or parts of the body can have differently sized bands than smaller or weaker patients or parts of the body.

The shapes described herein reduce the time needed to carry out a procedure compared to sutures, for two reasons. First, the total number of bands needed is lower than the number of sutures that would be needed, due to the increased physical size and tensile strength of the bands compared to a surgical suture. Second, the bands can be tightened by a relatively small tightening gun, rather than requiring access for a pair of hands as is the case with sutures. Therefore more people, such as a surgeon and one or more surgeon's assistants, can be involved in the tightening process than would be possible for tightening sutures.

The devices described herein are safer and more comfortable for physicians, reduce or eliminates the chance for breakage of the closure device, and permits tightening and re-tightening of the same ties, unlike sutures. The ties disclosed herein have a variety of shapes in different embodiments that are relatively low-profile and that will provide adequate holding strength for an incision to repair itself, while also being dissolvable for long-term patient safety and comfort.

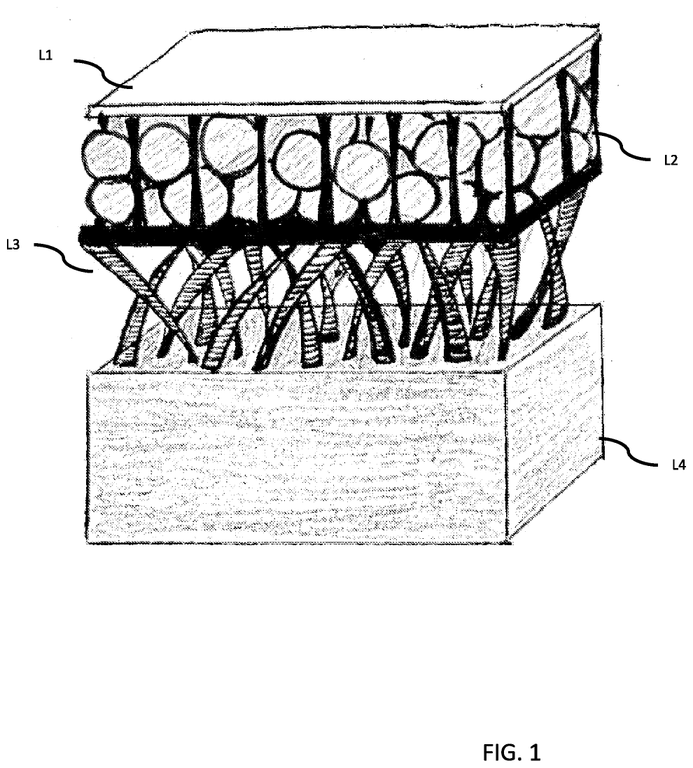

FIG. 1 is a detailed cross-sectional view of a fascia that can be held by devices described herein. As shown in FIG. 1, a typical portion of the body that can be held with the bands described herein includes a top layer L1 that is skin. The skin layer L1 is arranged above adipose tissues layer L2. The adipose tissues layer L2 is adjacent a layer L3 of loose connective tissues that connect the skin and adipose layers L1 and L2 to the muscle layer L4. However, a thin layer of deep fascia is interposed between adipose layer L2 and connective tissues layer L3, and it is this deep fascia that must be reconnected for proper healing after an incision.

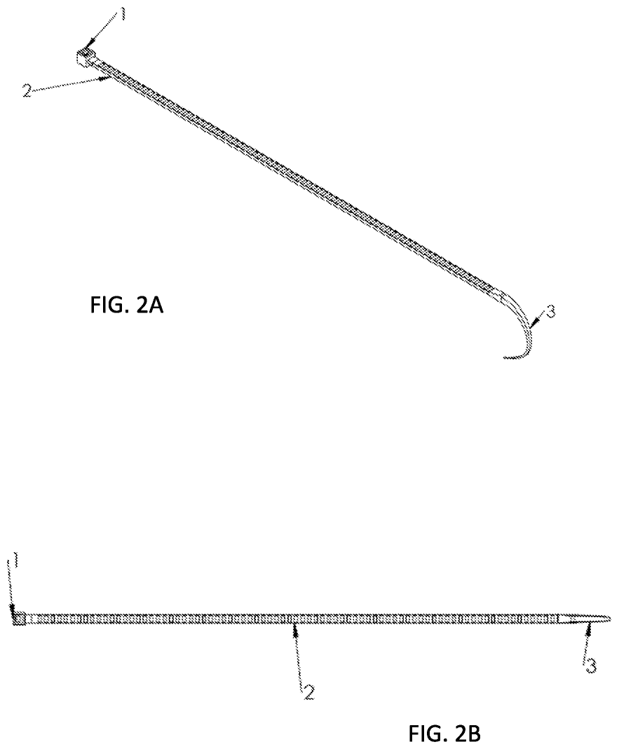

FIGS. 2A and 2B are perspective and top views, respectively, of a fastener according to a recessed-tooth embodiment. The fascia closing device illustrated in FIGS. 2A and 2B is a surgical fastener, where the locking teeth are recessed into the body 2. The zip tie has a locking mechanism 1 connected to the zip tie body 2 which is attached to a steel needle 3.

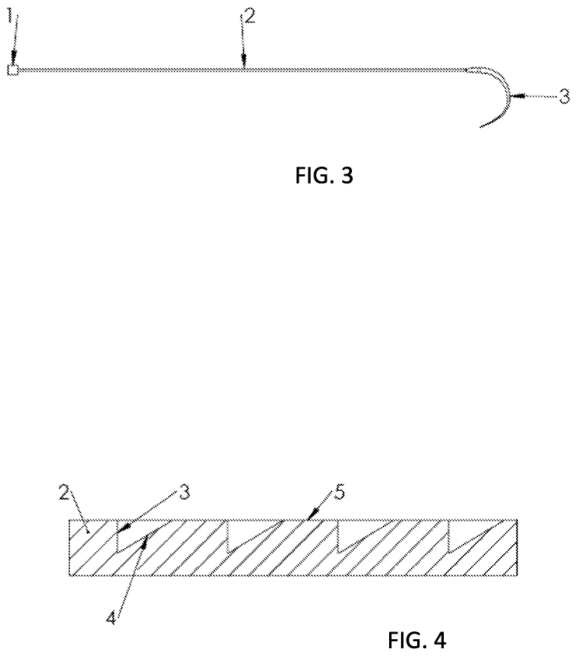

As shown in FIG. 3, the device has a smooth cross-section when the body 2 is viewed from the side. The smooth cross-section is due to the recessed nature of the teeth (not shown in FIG. 3) into the body 3. This recessed tooth structure facilitates easy sliding of the device through the fascia and other tissues without causing unnecessary trauma that a rough outer form factor could otherwise generate. Additionally, as shown in FIG. 3 the needle 3 has the largest cross-sectional width, such that pulling the body 2 through the opening created by the needle 3 should not cause any further stress on the surrounding tissue.

The zip tie body 2 of the embodiment of FIGS. 2A, 2B, and 3, as described above, has a solid body with triangular teeth recessed into it. This is shown in the detailed view of FIG. 4. The teeth have a vertical component 3 used in accordance with the locking mechanism to stop reverse motion and slanted face 4 to allow forward motion through the locking mechanism. These teeth are spaced evenly 5 over the top face of the body 2. As used herein, the term “vertical” and the like refer to the orientation on the page, and it should be understood that with respect to some other reference frame the teeth may not be vertically oriented whatsoever. In fact, as described in more detail below, the teeth may not even be oriented in the same direction as one another, as the entire device can be formed into a loop or other bent structure.

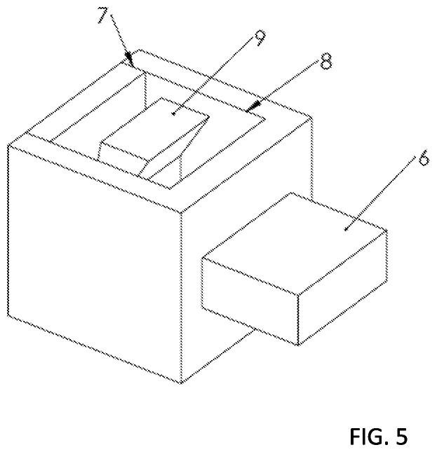

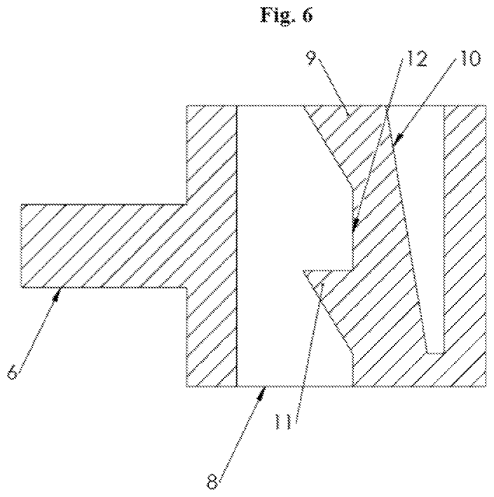

FIG. 5 shows a locking mechanism usable with the device described above with respect to FIGS. 2A, 2B, 3, and 4. In particular, the locking mechanism 1 of FIGS. 2A, 2B, and 3 can be substantially as depicted in FIG. 5.

The locking mechanism of FIG. 5 has an extension 6 that connects the outer shell 7 to the zip tie body. In embodiments, the extension 6 can be connected to the body 2 by mechanical friction fit, by melting or welding the components together, or other fastening mechanisms. In some embodiments (not shown), the extension 6 can be significantly larger than shown and include teeth, such that the extension 6 is the body 2. In such embodiments, it is possible for the device to be a single unitary device rather than having a locking mechanism (e.g., locking mechanism 1) that is formed separately from the body 2. In embodiments, a sleeve (not shown) or other structure can surround extension 6 to render the area around extension 6 smooth, rather than having sharp corners as depicted in FIG. 5.

In use, as shown in FIG. 6, the body (e.g., body 2) can be fed through the opening in the locking mechanism 8 and the tongue 9 is the pawl of the ratcheting mechanism. The slanted back of the tongue 10 allows for the tongue to displace as the zip tie is pulled through the locking mechanism, then snaps back to place once the zip tie teeth align with the pawl teeth 11. The pawl teeth 11 are spaced 12 equally to the teeth on the zip tie body. When the zip tie body is engaged with the pawl teeth, reverse motion is prevented by the interlocking bodies 13 of the teeth. The spacing between the teeth can be set based on the expected use. In some procedures it may be desirable to have more closely-spaced spacings 12, while for larger incisions the spacing 12 could be greater. In some embodiments, smaller spacing is desirable due to the higher accuracy of desired tension that it allows. In embodiments, spacing 12 can be between about 0.02 inches and about 0.05 inches.

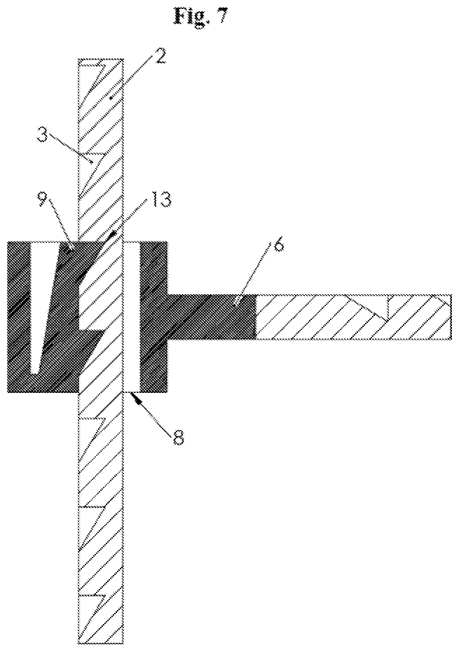

FIG. 7 is a cross sectional view of the body 2 of the recessed-style device of FIGS. 2A and 2B being fed through the locking mechanism to engage with tongue 9, illustrating how the tongue 9 of the locking mechanism fits in with the device body 2 to prevent backwards motion. As shown in FIG. 7, the interlocking bodies 13 of the teeth are engaged such that moving the body 2 upwards with respect to the page is possible, while moving the body 2 downward with respect to the page will cause tongue 9 to deform downwards and to the right, pinching the body 2 and preventing movement.

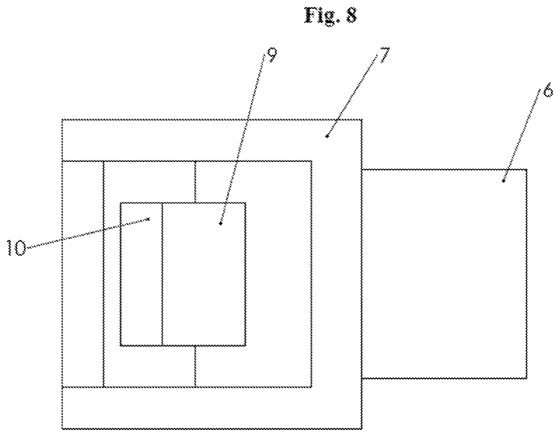

FIG. 8 is a plan view of the recessed-style locking mechanism component of FIGS. 2A and 2B, illustrating the relative body size of the major components described previously.

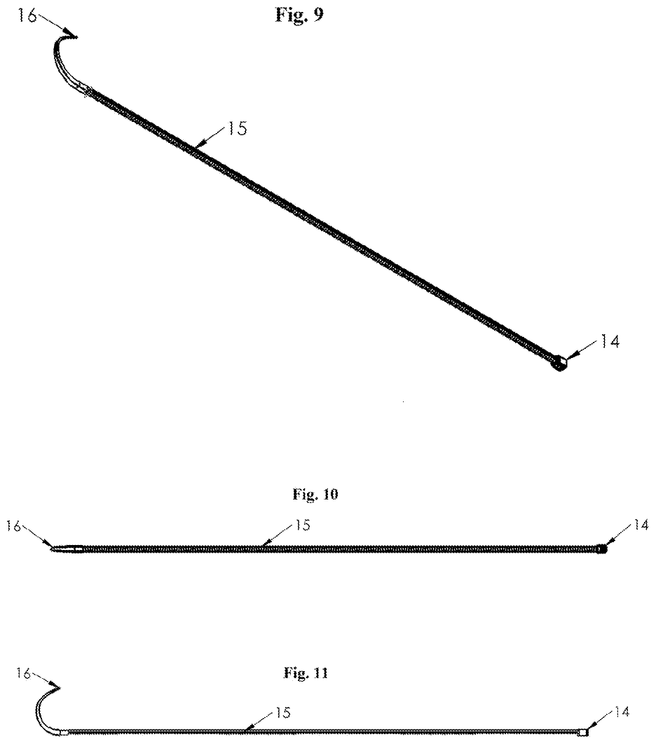

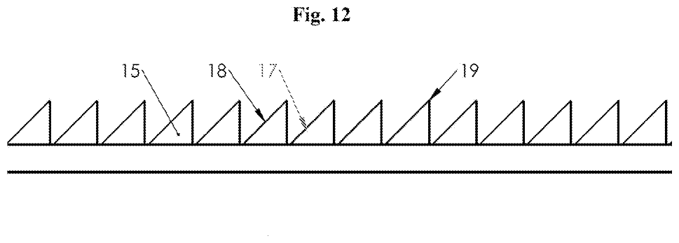

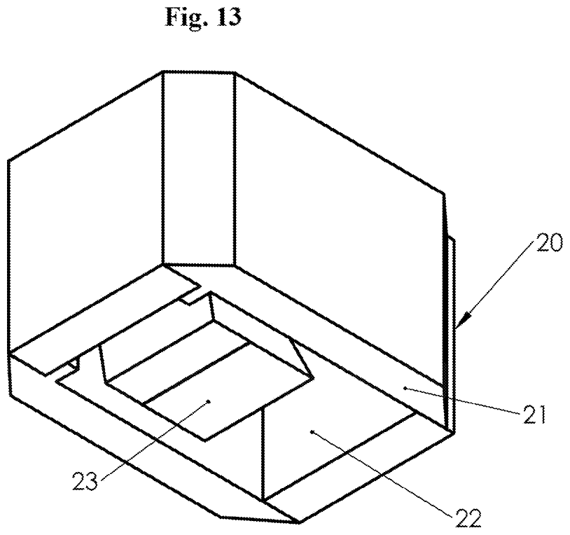

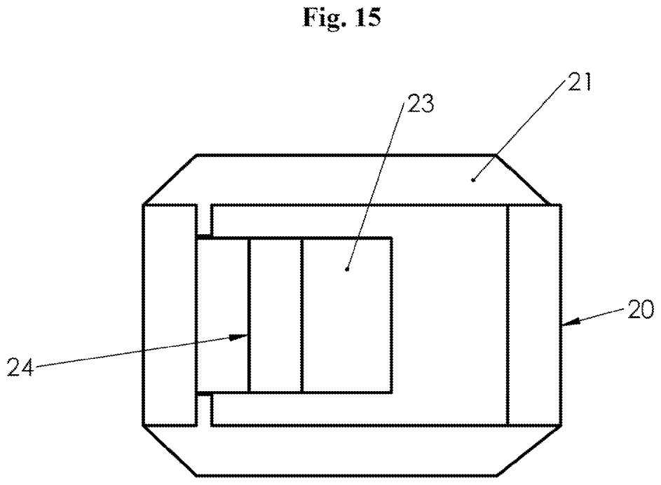

FIGS. 9-15 depict an exposed-style fascia closure device according to another embodiment. The terms “exposed-style” and “protruding teeth” are used to refer to the same types of devices throughout this disclosure. Unlike the embodiment described above with respect to FIGS. 2A-8, the teeth in this embodiment are not recessed. Exposed teeth 15 can grip to adjacent structures, including tissue, bone, or other devices.

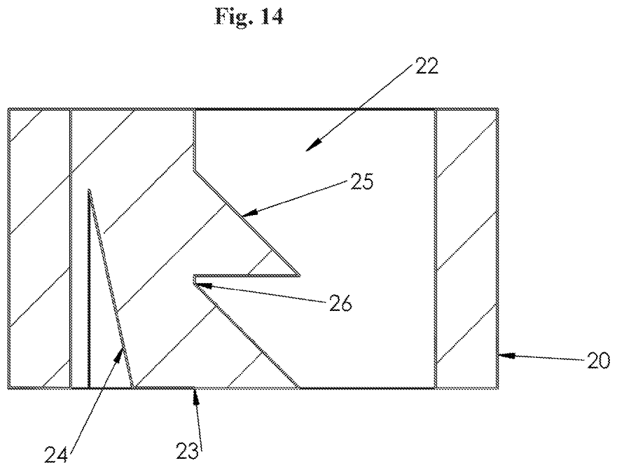

Like the recessed device, the exposed-style fascia device of FIGS. 9-15 has teeth 15 that rise at some slope 18 to a point 19. Extension 20 (similar to extension 6 described above) extends outwardly from the outer shell 21. Tongue 23 extends through an inner passage 22. The tongue 23 can be flexible so that surface 24 is parallel with a wall of the inner passage 22 when compressed, or angled outwards (as shown in FIG. 14) when not compressed.

The teeth can be made of a material 17 that is dissolvable in tissue so that the teeth 15 hold for a sufficient time for the fascia or other adjacent tissues to heal, before the structural integrity of the device is compromised due to the dissolving. In embodiments, the material can be a mix of polylactic acid and polyglycolic acid, such as a 90/10 copolymer blend. In alternative embodiments, the ratio of monomers could differ to be, for example, 999/1 to 1/999, such as about 80/20. Other monomers could be used that are soluble, such as polydioxanone, for example. In other embodiments, the material could be the same materials used currently for surgical sutures, such as VICRYL® or MONOCRYL® (poliglecaprone 25) materials.

The cross-section of the devices described herein can vary based on the expected load, the material or materials used in the construction of the device, and the cross-sectional area of the device at its narrowest point. The structure of the devices can therefore be modified to have different materials and/or minimum cross-sectional areas to support the needed load, while also remaining small enough to fully dissolve on the desired timeline and to avoid being uncomfortable or noticeable in the patient after the procedure.

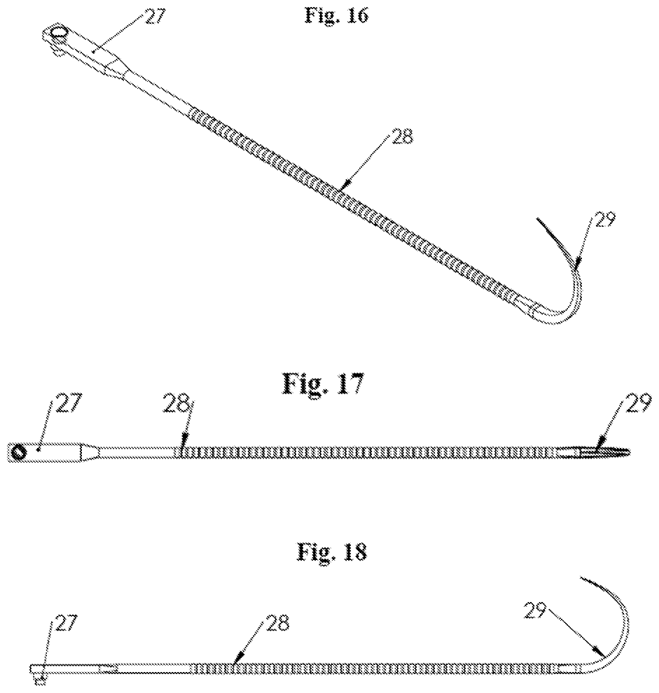

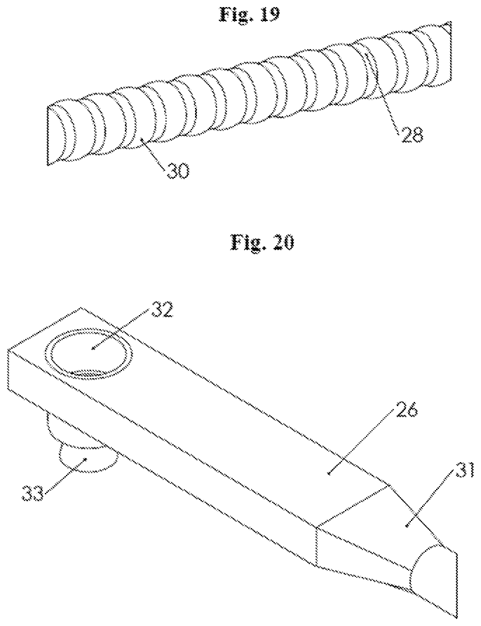

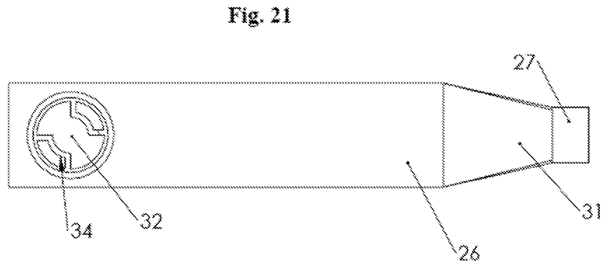

FIGS. 16-21 depict a fascia closing device having a circular cross-sectional shape according to another embodiment. A circular cross-section lacks sharp corners and edges, and can therefore be a more gentle tie for the patient. Additionally, due to the large ratio of volume to surface area, the circular cross-section will dissolve relatively slowly compared to a zip tie having another shape. Furthermore, a circular cross-section can be used easily with a more traditional needle shape (retaining a more spherical cross section). A circular cross section allows for easier bending in any direction since no bending direction has corners in the way. This allows for the tie to be placed non-perpendicularly within the incision without increased error risk from bending phenomena. Circular cross-sections can also provide more robust locking than devices in which the body must be aligned with the aperture of the locking portion.

The parts of the device shown in FIGS. 16-21 are similar to those of the previously-described embodiments, but modified the circular cross-section of the body. A modified locking mechanism 27 is configured to receive a ribbed portion 28 of the body. As shown in FIGS. 16 and 17, the locking mechanism 27 has a necked shape and a circular socket-like shape at a proximal end thereof (wherein the distal end is opposite the ribbed portion 28, at the needle 29). The ribbed portions 28 engage with teeth 25, which can be separated by gaps 26.

Ribbed portion 28 can include a series of ridges 30 opposite the necked portion 31 from the locking portion 27. The ridges can be received in an aperture 32 and locked in place by a deformable portion 33, as shown in FIG. 20. In various embodiments, the locking portion can be entirely internal to the locking mechanism, or it can partially or entirely protrude from the locking mechanism.

It should be understood that additional cross-sectional shapes could be appropriate for other embodiments. As described above, flat rectilinear ties and those with circular cross-sections are usable in a large number of embodiments. However, it may also be desirable to use ties that have a cross-sectional shape suitable for other purposes or locations within the patient.

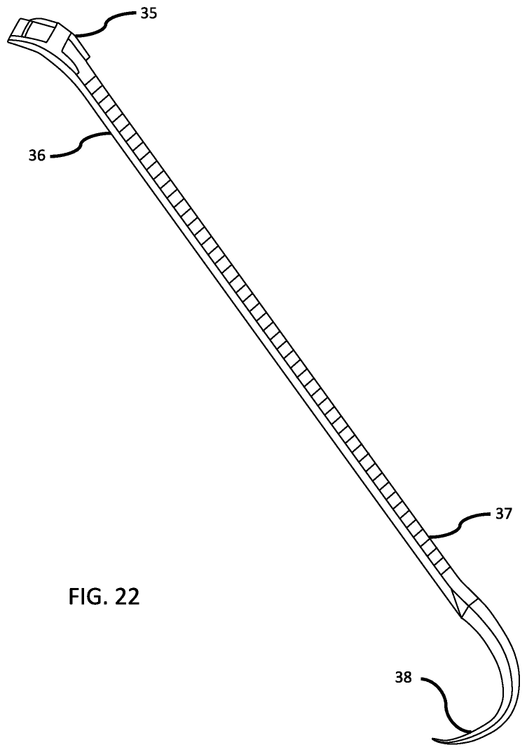

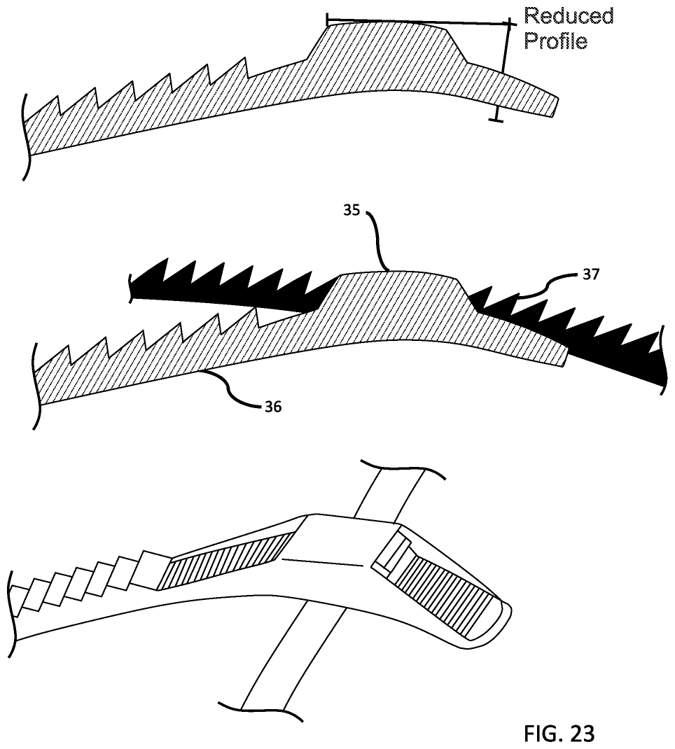

FIGS. 22 and 23 depict a side-loaded embodiment, which can have a reduced profile as shown in the top view of FIG. 23. As shown in FIG. 22, a locking mechanism 35 is coupled to a proximal portion 36 and a distal portion 37 that can be locked therein. The distal portion 37 is coupled to a needle 38. A closed loop can be formed with very low profile and without a sharp angle between the body and the body itself. In embodiments, the proximal portion 36 and the distal portion 37 can be parallel one another. In other embodiments, the form factor of locking mechanism 35 itself can have some radius of curvature corresponding to the expected size of the loop to be formed by the device.

The device in FIGS. 22 and 23 therefore provides a continuous curvature, or at least avoids the sharp angled curves of other types of zip ties. In embodiments, the radius of curvature can be between about 1 mm and about 10 mm, such as about ⅛ inch.

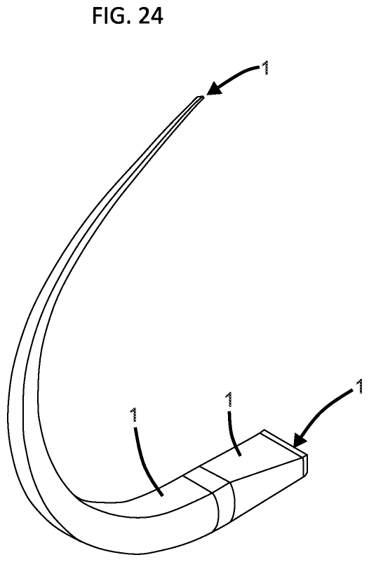

FIG. 24 is a perspective view of a needle that can be used with any of the embodiments described above. In various embodiments, the needle used can have a size and cross-section that matches with the body of the device. For example, a body with a rectilinear cross-section can correspond to a needle having a rectilinear cross-section, while a body with a circular or ovoid cross-section can correspond to a needle having a corresponding circular or ovoid cross-section. As described above, the cross-sectional size of the needle may be the same size or slightly larger than that of the body.

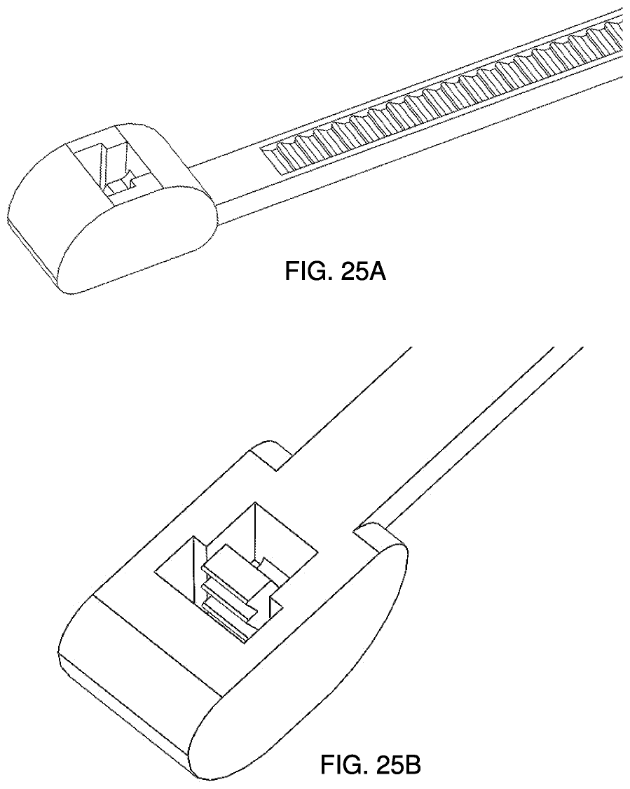

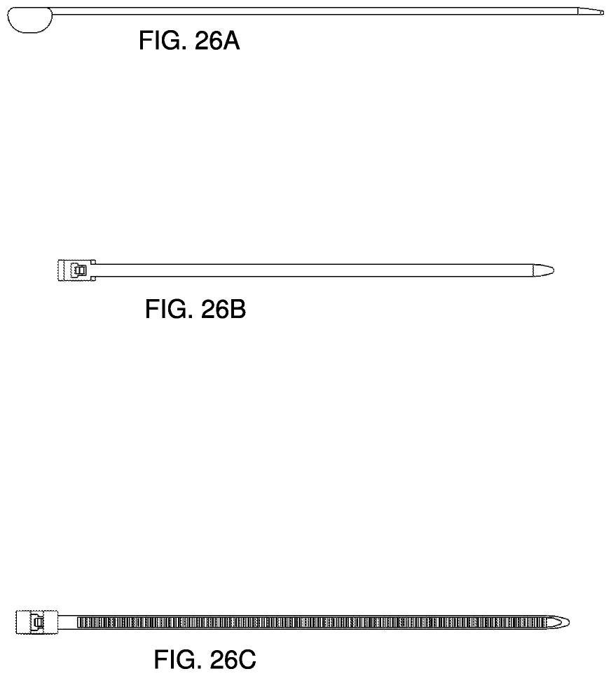

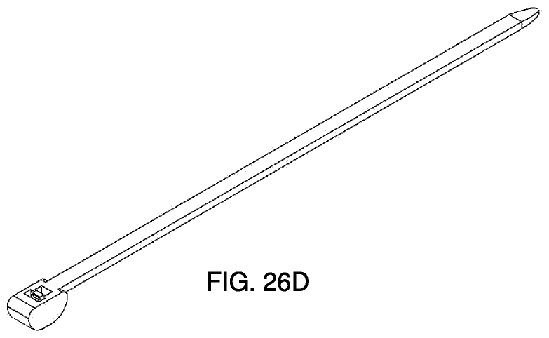

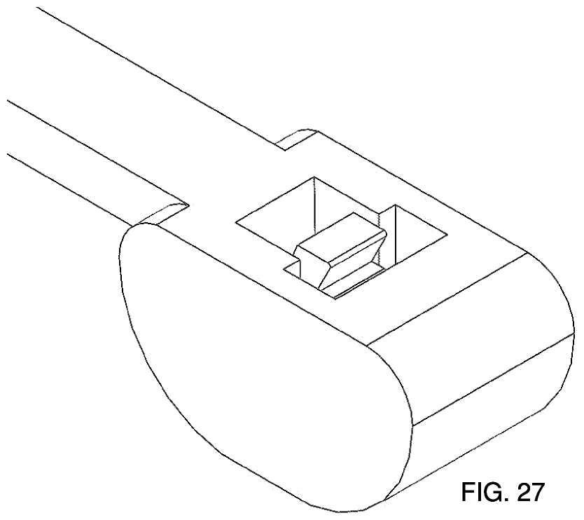

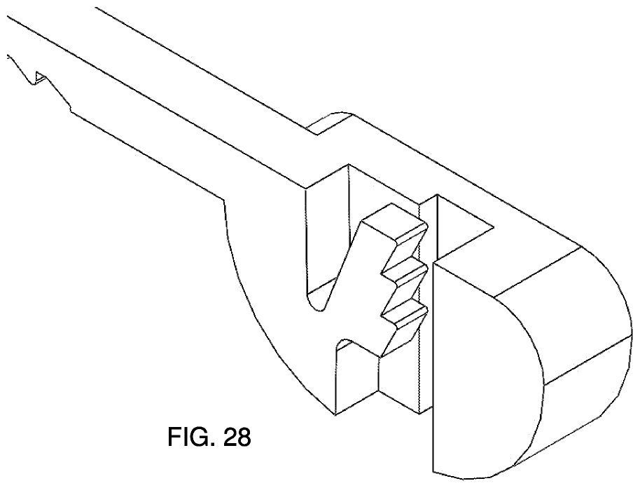

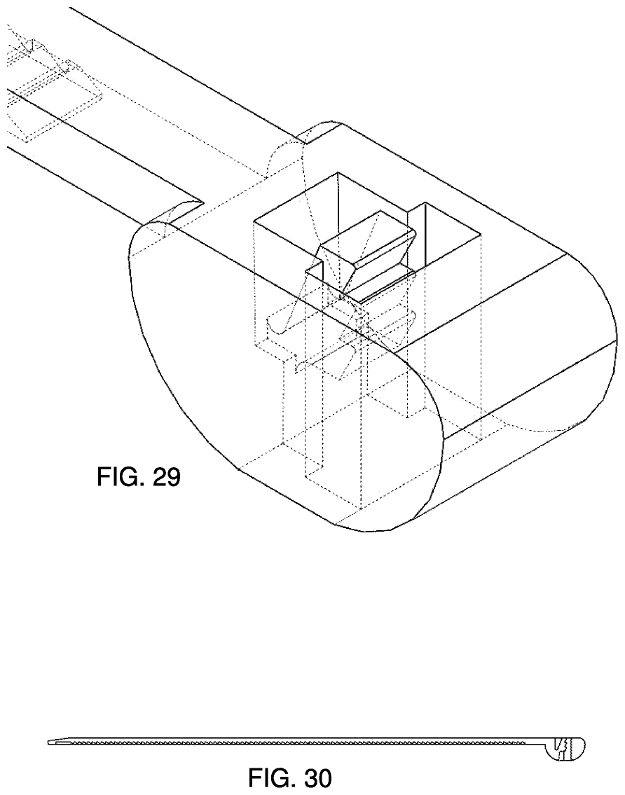

FIGS. 25A through 29 depict a fascia closure device with an alternative recessed style locking mechanism, according to embodiments.

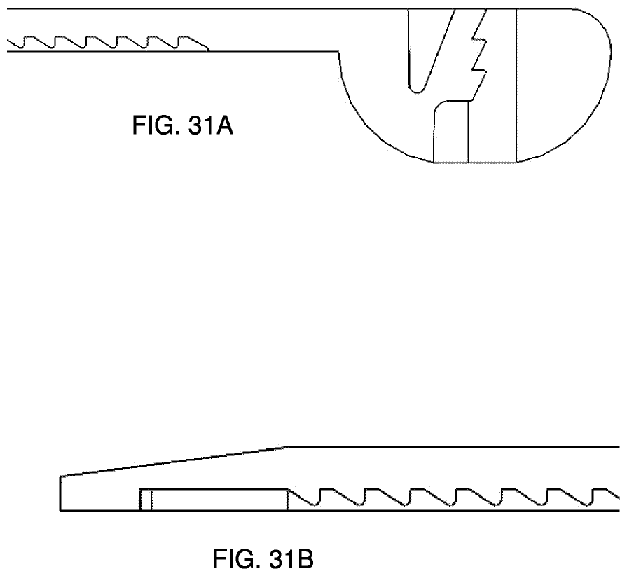

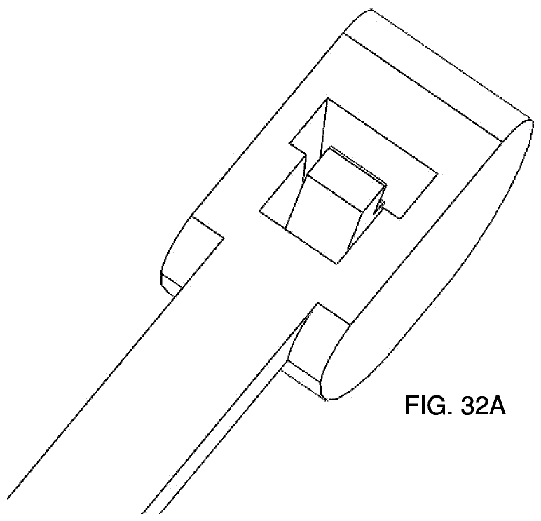

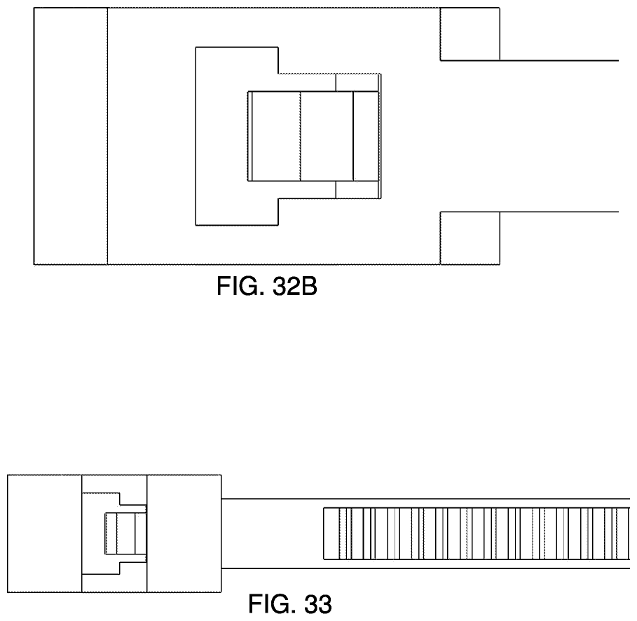

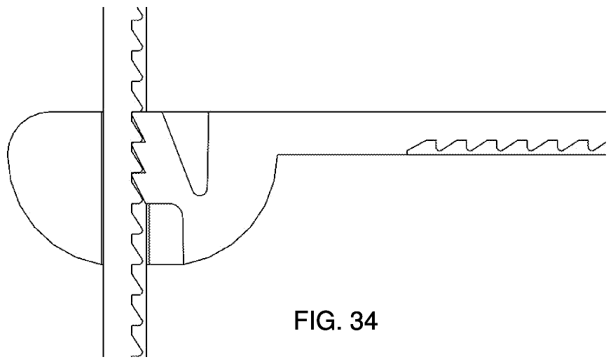

FIG. 30 through 34 depict a fascia closure device incorporating an alternative locking mechanism, according to embodiments. Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1.-22. canceled

23. A tie made of a biodegradable material, the tie configured to move from a delivery configuration to a locked configuration, the tie comprising:
a body extending along a longitudinal length and comprising a needle positioned near a distal end of the body and a locking mechanism positioned near a proximal end of the body; and
a series of recessed teeth positioned on a first face of the body and along at least a portion of the longitudinal length, each tooth of the series of teeth comprising a tooth slope falling into the body and a tooth edge rising substantially perpendicular to the body to a tooth point converging with a neighboring tooth slope,
wherein the locking mechanism is oriented to receive the distal end of the body when the tie is moved into the locked configuration, thereby forming a loop comprising the first face of the body oriented toward an internal volume of the loop.

24. The tie of claim 23, wherein the locking mechanism comprises:
an inner passage oriented to receive the distal end of the body; and
a locking engagement oriented to fit the series of recessed teeth on the first face of the body when the tie is in the locked configuration.

25. The tie of claim 23, wherein the proximal end of the locking mechanism further comprises a curvature.

26. The tie of claim 23, wherein the proximal end of the locking mechanism further comprises a curvature corresponding to the expected size of the loop formed by the tie in the locked configuration.

27. The tie of claim 23, wherein the needle comprises:
a diameter equal to or greater than a width of the body;
an outer radius configured to match with the first face of the body; and
an inner radius configured to match a second face of the body.

28. The tie of claim 23, wherein the locking mechanism further comprises a locking engagement arranged in the inner passage and responsive to an applied force of the tie passing through to bend from a hinge towards the sidewall of the locking mechanism, wherein the locking engagement is configured to mate with the series of recessed teeth on the first face of the body with a responsive force, fully grasping a tooth and restricting translational movement backwards through the inner passage.

29. The tie of claim 24, wherein the locking engagement comprises a tongue comprising one or more locking teeth on a first face, and a slanted back on a second face, wherein the slanted back of the tongue allows for the tongue to displace as the tip is pulled through the locking mechanism and snap back into place once the one or more locking teeth of the tongue align with the series of recessed teeth on the first face of the body.

30. The tie of claim 23, wherein a second face of the body oriented toward an external volume of the loop is smooth such that it minimizes trauma to surrounding tissues.

31. The tie of claim 23, wherein the body passed through the locking mechanism is ratcheted to hold a target tension.

32. The tie of claim 23, wherein the biodegradable material is biocompatible and bioabsortive in a body over a target time period; and
wherein the biodegradable material further comprises a compositional mixture of PLA and/or PLGA from 0:200 to 200:0.

33. The tie of claim 32, wherein the material enables the tie to move from the delivery configuration to form the loop of a target diameter in the locked configuration.

34. The tie of claim 32, wherein the material enables the tie to be cut with standard operating room scissors.

35. The tie of claim 30, wherein the tie further comprises an increased surface area on the first face of the body configured to be directly applied against a target and better supports surgical closure.

36. The tie of claim 23, wherein the tie is for use in surgical closure in a procedure selected from the following group: to stitch through fascial layers; to perform spinal closures to support the high tensile strength and force needed to hold closed the wound; cranial closures; laparoscopic surgery; endoscopic surgery; to stitch bones together; sternum closure in open heart and other applicable surgeries; emergency situations and applications by the military; and combinations thereof.

37. A method for surgical fascial closure, the method comprising
securing, with surgical tools, a tie made of a biodegradable material, wherein the tie is configured to move from a delivery configuration to a locked configuration, the tie comprising:
a body extending along a longitudinal length and comprising a needle positioned near a distal end of the body and a locking mechanism positioned near a proximal end of the body, and
a series of recessed teeth positioned on a first face of the body and along at least a portion of the longitudinal length, each tooth of the series of teeth comprising a tooth slope falling into the body and a tooth edge rising substantially perpendicular to the body to a tooth point converging with a neighboring tooth slope;
driving, with surgical tools, the tie through fascial layers; and
passing the distal end of the body through the locking mechanism oriented to receive the distal end of the body when the tie is moved into the locked configuration, thereby forming a loop comprising the first face of the body oriented toward an internal volume of the loop.

38. The method of claim 37, wherein passing the distal end of the body through the locking mechanism comprises passing the distal end of the body through an inner passage of the locking mechanism comprising a locking engagement configured to mate with the series of recessed teeth on the first face of the body with a responsive force, fully grasping a tooth and restricting translational movement backwards through the inner passage.

39. The method of claim 37, wherein the proximal end of the locking mechanism further comprises a curvature.

40. The method of claim 37, further comprising applying the first face of the body directly against a target such that a substantially smooth second face of the body is oriented towards surrounding tissues that minimizes trauma to surrounding tissues.

41. The method of claim 37, further comprising cutting the tie near the proximal end to be flush with the locking mechanism.

42. The method of claim 37, further comprising using an applied force to ratchet the tie by gradually moving the body of the tie through the locking mechanism such that the locking engagement mates from one tooth to the next tooth until a target tension is achieved; and
cutting the tie such that the needle is cut off or otherwise removed from the body of the tie in the locked configuration.
